(19) United States
(12) Patent Application Publication
(10) Pub. No.: US 2022/0079431 A1
(43) Pub. Date: Mar. 17, 2022

(54) METHOD FOR IMPROVING VISION

(71) Applicant: M Dan Siler, Box Elder, SD (US)

(72) Inventor: M Dan Siler, Box Elder, SD (US)

(21) Appl. No.: 17/021,200

(22) Filed: Sep. 15, 2020

(51) Int. Cl. A61B 3/04; A61B 3/032

(52) U.S. Cl. CPC A61B 3/04; A61B 3/032

(57) ABSTRACT

A method for improving vision utilizes a progressive series of various diopter eyeglasses, a vision chart or the like, and textual material. The first therapy commences with the person donning a first pair of eyeglasses having a magnifying diopter and the vision chart is positioned approximately 2 feet away from the person. The person then fixates upon the chart mark until the chart mark becomes visually clear. The person then moves farther away from the vision chart by approximately one foot and the person once again fixates upon the chart mark until the chart mark becomes visually clear. The second therapy commences with the person donning a first pair of eyeglasses having a low magnifying diopter and then reading textual material for several minutes. The person then replaces the low magnifying diopter eyeglass with a higher magnifying diopter eyeglass and once again reads the text for several minutes.

### TECHNICAL FIELD

The invention relates to a method for improving the vision in the eye.

### BACKGROUND OF INVENTION

Many people in the world suffer from poor or blurry vision. Blurry vision is the loss of sharpness of eyesight, making objects appear out of focus and hazy. The primary causes of blurry vision are refractive errors, which are commonly referred to as nearsightedness or farsightedness.

Farsightedness, presbyopia, hypermetropia, or hyperopia is another common refractive error that causes objects that are close in distance to appear blurred while objects that are far in distance to appear clear. Presbyopia is an age related condition in which the lens of the eye becomes stiffer and less flexible. Farsightedness is caused by a condition wherein the eye is vertically elongated in shape, causing the light passing through the lens and pupil is focused behind the retina, as shown in FIG. 3, instead of being focused on the retina, as shown in FIG. 1. Like myopia, hyperopia can be corrected with eyeglasses, contact lenses or refractive eye surgery. Again, these solutions are expensive and provide only a temporary solution as the eye continues to change over time.

Nearsightedness, or myopia is a common refractive error that causes objects in the distance to appear blurred while objects that are close in distance to appear clear. Nearsightedness is caused by a condition wherein the eye is flattened in shape, causing the light passing through the lens and pupil to be focused in front of the retina, as shown in FIG. 2, instead of being focused on the retina, as shown in FIG. 1. Eyeglasses, contact lenses and refractive surgery such as LASIK (laser assisted in situ keratomileusis) and PRK (laser assisted photorefractive keratectomy) are the most common ways to correct nearsightedness. However, these solutions are expensive and provide only a temporary solution as the eye continues to change over time.

If the eye shape is incorrect to a minor degree, these conditions may be overcome with a healthy lens of the eye, as the ciliary eye muscles reshapes the lens in order to refocus the eye. However, as one ages, presbyopia ensues, the eye lens becomes less flexible, stiffer, and more embrittled, thereby limiting the ability of the ciliary eye muscles to reshape the lens to correct the vision.

Accordingly, there is a need in the art a method to provide improved vision without the need of corrective lenses or surgery. It is to the provision of such therefore that the present invention is primarily directed.

### SUMMARY OF THE INVENTION

In a preferred form of the invention, a method for improving vision of a person comprises the steps of (A) providing an plurality of different diopter eyeglasses, (B) providing reading material having text thereon, (C) having the person wear the lowest diopter eyeglasses for a plurality of minutes while reading the text of the reading material to cause eye strain, (D) removing the lowest diopter eyeglasses and replacing the lowest diopter eyeglasses with the next higher diopter eyeglasses, and (E) having the person read the text of the reading material for a plurality of minutes to cause eye strain created by the next higher diopter eyeglasses.

In another preferred form of the invention, a method of improving the vision of a person comprising the steps of (A) providing a chart having a chart mark, (B) providing a pair of diopter eyeglasses, (C) positioning the chart at least 2 feet from the person, (D) having the person fixate upon the chart mark for at least one minute while donning the pair of magnifying diopter eyeglasses, (E) moving the person to a position at least 3 feet from the chart, and (F) having the person fixate upon the chart mark for at least one minute while donning the pair of magnifying diopter eyeglasses.

### BRIEF DESCRIPTION OF DRAWINGS

FIG. 1 is a schematic side view of an eye showing normal vision.

FIG. 2 is a schematic side view of an eye showing nearsighted vision.

FIG. 3 is a schematic side view of an eye showing farsighted vision.

FIG. 4 is a schematic view of the method embodying principles of the invention in a preferred form.

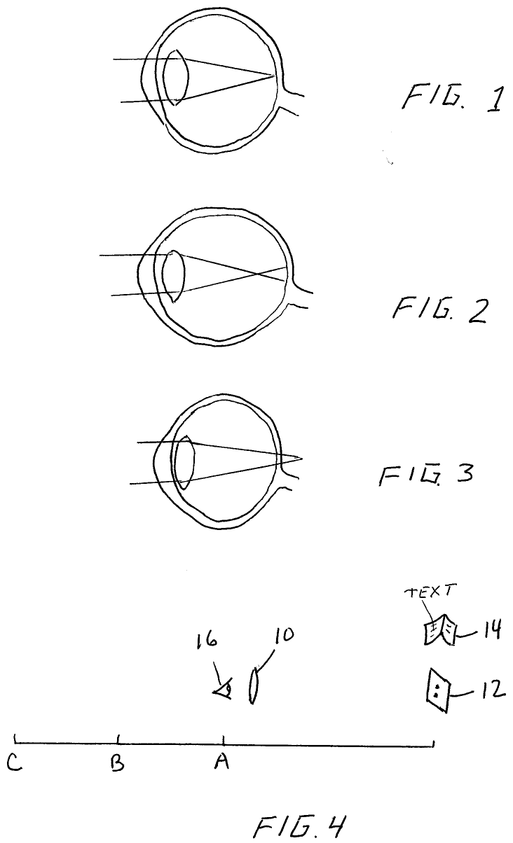

### DETAILED DESCRIPTION

With reference next to the drawings, there is shown a method of improving vision in a preferred form of the invention. The method of improving vision utilizes a progressive series of various magnifying diopter eyeglasses 10, a vision chart 12 or the like, and textual material 14. The eyeglasses 10 should be clean and round in shape to provide the best overall line of sight, rather than a narrow lens which may limit the line of sight through the lens in addition to restricting the peripheral vision. The method of improving vision consist of two separate steps, therapies, or exercises. The results include, but are not limited to, increased lens flexibility.

The first therapy should be conducted once a month, but may be conducted less often depending on the results. The person commences the therapy by donning a first pair of eyeglasses 10 having a magnifying diopter, for example a 1.25 diopter eyeglasses. A high contrasting vision chart 12, having indicia, markings, symbols, etc., referenced hereinafter simply as “chart mark”, is positioned approximately and at least 2 feet away from the person, as shown in FIG. 4 as position A. With both eyes open, the person's initial vision will be blurred and as such the person must fixates upon the chart mark until the chart mark becomes visually clear, which should take at least one minute.

The person then moves farther away from the vision chart 12 by approximately one foot, so that there is now at least three foot distance between the vision chart 12 and the person, as shown in FIG. 4 as position B. The person's eyes will not be able to immediately focus on the chart mark. Therefore, the person once again fixates upon the chart mark until the chart mark becomes visually clear. If the person is able to successfully focus upon the vision chart 12, the person should move another foot back from the vision chart 12, as shown in FIG. 4 as position C.

Should the person be unable to obtain focus on the chart mark, the person moves forward one foot so that the person is once again positioned at the previous position wherein the eye 16 could focus on the vision chart 12. Now, the person fixates upon the chart mark again until it is visually clear or in focus. The person then stares at the chart mark keeping it in focus for several minutes. Because of the person's movement, the first therapy is best accomplished while standing to make such movements quick and convenient.

It is believed that the first therapy aids in increasing the strength of the ciliary eye muscles. As a result of such improvements in the physiology, the eye provides faster ciliary muscle response times resulting in quicker focusing on an object by the brain, which may be referenced as the accommodative amplitude or amplitude of accommodation which is the adjustment of the eye 16 in seeing objects at different distances. For it is the ciliary muscles which contracts or relaxes to reshape the lens in refocusing the eye 16. This also results in an increase in the visual acuity, which is the sharpness of vision, and faster visual acuity response times and lower refractive errors. The eye strain and resulting stain or movement on the lens caused by the ciliary eye muscles may be considered to be a hyper-flexing or over-flexing of the eye lens. As a result of this hyper-flexing, the lens is believed to regain increased flexibility and elasticity.

The second therapy should be conducted daily or at least weekly. The person commences the therapy by donning a first pair of eyeglasses 10 having a low magnifying diopter, for example a 1.25 diopter eyeglasses. Textual material 14, such as a book, magazine, computer screen, etc., is provided to the person, referenced hereinafter simply as “text”. With both eyes open, the person then fixates upon or reads the text for several minutes. The magnifying diopter eyeglasses 10 causes an eye strain to overcome the blurring caused by the magnifying diopter eyeglasses 10. The person then reads for a period of time, several minutes or longer, after which the person replaces the low magnifying diopter eyeglass with a higher magnifying diopter eyeglass (medium magnifying eyeglasses), such as a 1.50 diopter eyeglass. The person then once again fixates upon or reads the text for several minutes. Again, the magnifying diopter eyeglasses 10 causes an eye strain to overcome the blurring caused by the magnifying diopter.

Next, the person then reads for a period of time, several minutes or longer, after which the person replaces the medium magnifying diopter eyeglass with a higher magnifying diopter eyeglass (high magnifying eyeglasses), such as a 1.75 diopter eyeglass. The person then once again fixates upon or reads the text for several minutes. Once again, the magnifying diopter eyeglasses 10 causes an eye strain to overcome the blurring caused by the magnifying diopter.

The person may continue to increase the magnifying diopter of additional eyeglasses 10 if that person is able to regain a focus on the text with such increases.

It is believed that the eye strain created by the second therapy also aids in increasing the strength of the ciliary eye muscles. This also makes improvements in the physiology, the eye provides faster ciliary muscle response times resulting in quicker focusing on an object by the brain, which may be referenced as the accommodative amplitude or amplitude of accommodation which is the adjustment of the eye in seeing objects at different distances. Again, this also results in an increase in the visual acuity, which is the sharpness of vision, and faster visual acuity response times and lower refractive errors. The eye strain and resulting stain or movement on the lens caused by the ciliary eye muscles may be considered to be a hyper-flexing or over-flexing of the eye lens. As a result of this hyper-flexing, the lens is believed to regain increased flexibility and elasticity.

The present method for improving farsightedness utilizes a biconvex or plano-convex corrective lens. The method may utilize a biconcave or plano-concave lens to correct nearsightedness.

From the forgoing description, there has been shown a method for improving vision of a person comprises the steps of (A) providing an plurality of different diopter eyeglasses, (B) providing reading material having text thereon, (C) having the person wear the lowest diopter eyeglasses for a plurality of minutes while reading the text of the reading material to cause eye strain, (D) removing the lowest diopter eyeglasses and replacing the lowest diopter eyeglasses with the next higher diopter eyeglasses, (E) having the person read the text of the reading material for a plurality of minutes to cause eye strain created by the next higher diopter eyeglasses, (F) removing the next higher diopter eyeglasses and replacing the next higher diopter eyeglasses with an even higher diopter eyeglasses, and (G) having the person read the text of the reading material for a plurality of minutes to cause eye strain created by the even higher diopter eyeglasses.

It has also been shown that a method of improving the vision of a person comprising the steps of (A) providing a chart having a chart mark, (B) providing a pair of diopter eyeglasses, (C) positioning the chart at least 2 feet from the person, (D) having the person fixate upon the chart mark for at least one minute while donning the pair of magnifying diopter eyeglasses, (E) moving the person to a position at least 3 feet from the chart, (F) having the person fixate upon the chart mark for at least one minute while donning the pair of magnifying diopter eyeglasses, (G) providing a second higher diopter pair of magnifying diopter eyeglasses having a higher diopter than the first higher diopter pair of magnifying diopter eyeglasses, (H) donning the third magnifying diopter eyeglasses by the person being treated for weak vision, and (I) having the person fixate upon the text until the text becomes clear.

Variations and modifications of the method disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

1. A method for improving the vision of a person comprises providing a first magnifying diopter eyeglasses, donning the first magnifying diopter eyeglasses by a person being treated for weak vision to cause the eye to have initially blurred vision, having the person fixate upon text until the text becomes clear, providing a second magnifying diopter eyeglasses having a higher diopter than the first magnifying diopter eyeglasses, donning the second magnifying diopter eyeglasses by the person being treated for weak vision to cause the eye to have initially blurred vision again, and having the person fixate upon the text until the text becomes clear.

2. The method of improving the vision of a person of claim 1 further comprising providing a third magnifying diopter eyeglasses having a higher diopter than the second magnifying diopter eyeglasses, donning the third magnifying diopter eyeglasses by the person being treated for weak vision, and having the person fixate upon the text until the text becomes clear.

3. The method of improving the vision of a person of claim 1 further comprising providing a chart having a chart mark, positioning the chart at least 2 feet from the person, and having the person fixate upon the chart mark for at least one minute while donning either one of the first magnifying diopter eyeglasses or second magnifying diopter eyeglasses until the chart mark becomes clear.

4. The method of improving the vision of a person of claim 3 further comprising having the person move to a position at least 3 feet from the chart, and having the person fixate upon the chart mark for at least one minute while donning either one of the first magnifying diopter eyeglasses or second magnifying diopter eyeglasses until the chart mark becomes clear.

5. The method of improving the vision of a person of claim 1 wherein the method is repeated at least one time a week.

6. A method for improving vision of a person comprises the steps of:
(A) providing an plurality of different diopter eyeglasses;
(B) providing reading material having text thereon;
(C) having the person wear the lowest diopter eyeglasses for a plurality of minutes while reading the text of the reading material to cause eye strain until the text becomes clear;
(D) removing the lowest diopter eyeglasses and replacing the lowest diopter eyeglasses with the next higher diopter eyeglasses, and
(E) having the person read the text of the reading material for a plurality of minutes to cause eye strain created by the next higher diopter eyeglasses until the text becomes clear.

7. The method of improving the vision of a person of claim 6 further comprising the step of:
(F) removing the next higher diopter eyeglasses and replacing the next higher diopter eyeglasses with an even higher diopter eyeglasses, and
(G) having the person read the text of the reading material for a plurality of minutes to cause eye strain created by the even higher diopter eyeglasses until the text becomes clear.

8. The method of improving the vision of a person of claim 6 further comprising providing a chart having a chart mark, positioning the chart at least 2 feet from the person, and having the person fixate upon the chart mark for at least one minute while wearing one of the plurality of different diopter eyeglasses until the chart mark becomes clear.

9. The method of improving the vision of a person of claim 8 further comprising having the person move to a position at least 3 feet from the chart, and having the person fixate upon the chart mark for at least one minute while wearing one of the plurality of different diopter eyeglasses until the chart mark becomes clear.

10. The method of improving the vision of a person of claim 6 wherein the method is repeated at least one time a week.

11. A method of improving the vision of a person comprising the steps of:
(A) providing a chart having a chart mark;
(B) providing a pair of diopter eyeglasses;
(C) positioning the chart at least 2 feet from the person;
(D) having the person fixate upon the chart mark for at least one minute while donning the pair of magnifying diopter eyeglasses until the chart mark becomes clear;
(E) moving the person to a position at least 3 feet from the chart, and
(F) having the person fixate upon the chart mark for at least one minute while donning the pair of magnifying diopter eyeglasses until the chart mark becomes clear.

12. The method of improving the vision of claim 11 further comprising the steps of:
(G) donning the pair of diopter eyeglasses by a person being treated for weak vision to cause the eye to have initially blurred vision;
(H) having the person fixate upon text until the text becomes clear;
(I) providing a first higher diopter pair of magnifying diopter eyeglasses having a higher diopter than the diopter of the pair of magnifying diopter eyeglasses recited in step (G),
(J) donning the first higher diopter pair of magnifying diopter eyeglasses by the person being treated for weak vision to cause the eye to have initially blurred vision again, and
(K) having the person fixate upon the text while wearing the first higher diopter pair of magnifying diopter eyeglasses until the text becomes clear.

13. The method of improving the vision of a person of claim 12 further comprising the step of:
(L) providing a second higher diopter pair of magnifying diopter eyeglasses having a higher diopter than the first higher diopter pair of magnifying diopter eyeglasses,
(M) donning the third magnifying diopter eyeglasses by the person being treated for weak vision, and
(N) having the person fixate upon the text until the text becomes clear.
